(19) United States
(12) Patent Application Publication
(10) Pub. No.: US 2023/0233456 A1
(43) Pub. Date: Jul. 27, 2023

(54) COMPOSITIONS AND METHODS FOR TREATING CORONAVIRUS

(71) Applicant: PER OS BIOSCIENCES, LLC, Hunt Valley, MD (US)

(72) Inventors: Robert ESTEY, Reisterstown, MD (US); Lisa BACHMANN, Forest Hill, MD (US); William O. BRISBEN, Hobe Sound, FL (US)

(21) Appl. No.: 17/996,545

(22) PCT Filed: Mar. 21, 2022
(86) PCT No.: PCT/US2022/021123

§ 371 Date: Oct. 19, 2022

Related U.S. Application Data
(60) Provisional application No. 63/165,898, filed on Mar. 25, 2021.

(51) Int. Cl. A61K 9/00; A61K 9/68; A61K 9/20; A61K 45/06; A61K 31/55; A61K 31/401; A61K 31/675; A61K 31/472; A61K 31/722; A61K 33/30; A61K 33/18; A61K 31/155; A61K 31/44; A61K 31/7016; A61K 36/59; A61K 31/49; A61K 31/79; A61K 33/40; A61K 38/16; A61K 31/724

(52) U.S. Cl. CPC A61K 9/0056; A61K 9/0058; A61K 9/2013; A61K 9/2054; A61K 9/2018; A61K 45/06; A61K 31/55; A61K 31/401; A61K 31/675; A61K 31/472; A61K 31/722; A61K 33/30; A61K 33/18; A61K 31/155; A61K 31/44; A61K 31/7016; A61K 36/59; A61K 31/49; A61K 31/79; A61K 33/40; A61K 38/168; A61K 31/724

(57) ABSTRACT

Oral formulations include anti-viral agents. These oral formulations are manufactured in various permutations such as a chewing gum or lozenge which incorporate anti-viral agents for protecting an individuals body from viruses.

### CROSS REFERENCE TO RELATED APPLICATIONS

This application claims the benefit of priority under 35 U.S.C. § 119(e) to U.S. Provisional Application No. 63/165,898 filed Mar. 25, 2021. The entire contents of this application is incorporated herein by reference in its entirety.

### FIELD

The present disclosure relates to the composition and manufacture of an oral composition containing ingredients that prevent viruses from binding to the angiotensin-converting enzyme (ACE)-2 and, more specifically, a chewing gum or lozenge incorporating anti-viral active compounds either individually or in combination.

### BACKGROUND

Coronaviruses are a group of enveloped viruses with non-segmented, single stranded, and positive sense RNA genomes. While most coronaviruses cause mild respiratory illnesses, epidemics have been caused by the severe acute respiratory syndrome virus SARS-CoV-1 in 2003, and the SARS-CoV-2 virus beginning in 2020. SARS-CoV-1 spread to over two dozen countries affecting more than 8,000 individuals with nearly 10% mortality. The 2020 SARS-CoV-2 pandemic rapidly spread globally affecting millions of people with a corresponding mortality count of millions of individuals (Srinivasan 2020).

### SUMMARY

There is an unmet need for a safe, efficacious and cost-effective solution to inactivating Coronaviruses in the oral cavity before the viruses can spread disease. Accordingly, embodiments are directed to compositions which deliver therapeutically effective amounts of compounds directly into the oral cavity to a subject in need thereof. In particular, the oral compositions comprise chewing gum and lozenges which deliver active anti-viral agents directly into the oral cavity.

### Definitions

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

As used in this specification and the appended claims, the term “or” is generally employed in its sense including “and/or” unless the content clearly dictates otherwise.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value or range. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

As used herein, “active” is defined as the agent or agents that provide a therapeutic effect.

As used herein, the term “agent” or “active agent” or “compound” is meant to encompass any molecule, chemical entity, composition, drug, therapeutic agent, chemotherapeutic agent, or biological agent capable of preventing, ameliorating, or treating a disease or other medical condition. The term includes small molecule compounds, peptides, organic or inorganic molecules, natural or synthetic compounds and the like. An agent can be assayed in accordance with the methods of the invention at any stage during clinical trials, during pre-trial testing, or following FDA-approval. In certain embodiments, the active agent is hemp oil. In other embodiments, the agent is an extract of industrial hemp derived from a cultivar comprising a cannabidiol (CBD) and expressing low levels of tetrahydrocannabinol (THC). In other embodiments, the agent comprises a cannabidiol (CBD), tetrahydrocannabinol (THC) or combinations thereof.

As used herein, the term “cannabinoid” refers to a chemical compound that shows direct or indirect activity at a cannabinoid receptor. There are two main cannabinoid receptors, CB1 and CB2. Other receptors that research suggests have cannabinoid activity include the GPR55 and GPR 18 receptors. The term “phytocannabinoid” refers to cannabinoids that occur in a plant species or are derived from cannabinoids occurring in a plant species. Examples of cannabinoids include, but are not limited to, Tetrahydrocannabinol (THC), Cannabidiol (CBD), Cannabinol (CBN), Cannabigerol (CBG), Cannabichromene (CBC), Cannabicyclol (CBL), Cannabivarin (CBV), Tetrahydrocannabivarin (THCV), Cannabidivarin (CBDV), Cannabichromevarin (CBCV), Cannabigerovarin (CBGV), Cannabigerol Monomethyl Ether (CBGM). It should be understood, that compounds used in the art of pharmaceutics generally serve a variety of functions or purposes. Thus, if a compound named herein is mentioned only once or is used to define more than one term herein, its purpose or function should not be construed as being limited solely to that named purpose(s) or function(s).

As used herein, the term “chewing gum” refers to a flavored or non-flavored substance intended for chewing. The term as used herein also includes bubble gum and confectionery products containing chewing gum. In certain embodiments, chewing gum forms include, but are not limited to, tablets, sticks, solid balls, hollow balls, cut and wrap, and pellets or pillows.

As used herein a “derivative” is: a chemical substance that is related structurally to a first chemical substance and theoretically derivable from it; a compound that is formed from a similar first compound or a compound that can be imagined to arise from another first compound, if one atom of the first compound is replaced with another atom or group of atoms; a compound derived or obtained from a parent compound and containing essential elements of the parent compound; or a chemical compound that may be produced from first compound of similar structure in one or more steps.

As defined herein, a “therapeutically effective” amount of a compound or agent (i.e., an effective dosage) means an amount sufficient to produce a therapeutically (e.g., clinically) desirable result. The compositions can be administered from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compounds of the invention can include a single treatment or a series of treatments.

As defined herein, an “effective” amount of a compound or agent (i.e., an effective dosage) means an amount sufficient to produce a (e.g., clinically) desirable result.

As used herein, a “pharmaceutically acceptable” component/carrier etc. is one that is suitable for use with humans and/or animals without undue adverse side effects (such as toxicity, irritation, and allergic response) commensurate with a reasonable benefit/risk ratio.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health. A disease or disorder is “alleviated” if the severity of a symptom of the disease or disorder, the frequency with which such a symptom is experienced by a patient, or both, is reduced.

The terms “patient” or “individual” or “subject” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters, and primates.

“Treatment” is an intervention performed with the intention of preventing the development or altering the pathology or symptoms of a disorder. Accordingly, “treatment” refers to both therapeutic treatment and prophylactic or preventative measures. “Treatment” may also be specified as palliative care. Those in need of treatment include those already with the disorder as well as those in which the disorder is to be prevented. Accordingly, “treating” or “treatment” of a state, disorder or condition includes: (1) preventing or delaying the appearance of clinical symptoms of the state, disorder or condition developing in a human or other mammal that may be afflicted with or predisposed to the state, disorder or condition but does not yet experience or display clinical or subclinical symptoms of the state, disorder or condition; (2) inhibiting the state, disorder or condition, i.e., arresting, reducing or delaying the development of the disease or a relapse thereof (in case of maintenance treatment) or at least one clinical or subclinical symptom thereof; or (3) relieving the disease, i.e., causing regression of the state, disorder or condition or at least one of its clinical or subclinical symptoms. The benefit to an individual to be treated is either statistically significant or at least perceptible to the patient or to the physician.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

It should be understood that numerous specific details, relationships, and methods are set forth to provide a full understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or concurrently with other acts or events. Furthermore, not all illustrated acts or events are required to implement a methodology in accordance with the present invention.

Other aspects are described infra.

### DETAILED DESCRIPTION

Provided herein, is an innovative chewing gum that contains safe and effective active ingredients with properties that either prevent viruses from spreading throughout a person's body, or to another person. The regular use of the gums embodied herein would serve as a prophylactic solution at the main point of entry for a virus into the body before the virus can spread disease. The target population is any human being.

Specifically, Coronaviruses use the angiotensin converting enzyme (ACE-2) as the host receptor for target cell entry. The extent and distribution of ACE-2 has been associated with the clinical symptoms of coronavirus disease. ACE-2 is abundantly expressed in oral mucosa. Once firmly established in the oral cavity and replicating, Coronaviruses are:
easily spread to other ACE-2 expressing epithelial cells in the lungs, intestines, kidneys, and colon with the potential for causing significant health problems which can be severe and deadly.
easily expelled from the oral cavity via breathing, coughing or sneezing thus spreading the virus quickly to other people in close proximity.

ACE-2 is a protein on the surface of many cell types. It is an enzyme that generates small proteins (by cutting up the larger protein angiotensinogen) that then go on to regulate functions in the cell. Using the spike-like protein on its surface, Coronaviruses bind to ACE-2 (like a key being inserted into a lock) prior to entry and infection of cells. Hence, ACE-2 acts as a cellular doorway (a receptor) for the virus (Sriam, 2021).

Chewing gum. In certain embodiments, the composition is a chewing gum which releases the active agent(s) during chewing. A suitable chewing gum base comprises one or more constituents including elastomers for elasticity, resins to act as binders and softeners, plasticizers to render the elastomer soft to ensure thorough blending of the gum base and flavors during shelf life. The method for manufacture of a chewing gum is exemplified in U.S. Pat. No. 9,744,128 issued Aug. 29, 2017, the contents of which are incorporated herein by reference, in its entirety. Briefly, the method comprises initially heating the gum base in ovens to melt the gum base to an internally measured temperature between 140-160° F. The ingredients, including the one or more active ingredients are combined in a mixer. The melted gum base is added to the mixer and cooled to produce a particulate mixture. The temperature of the gum base exceeds that of the mixer when first introduced, but as mixing continues it cools quickly to room temperature and forms rock-sized granular pieces. These granular pieces are then conditioned for a period of time which allows the granular pieces to dry slightly and complete the crystallization process. The pieces are conditioned for at least about 6 hours at a temperature not greater than about 75° F. and about 60% relative humidity. The pieces are then ground into a powder at room temperature with tableting excipients, and tableted. This process preserves the efficacy of the active ingredient or ingredients by avoiding exposure to high heat and extreme cold, mainly during milling that can otherwise degrade the active ingredient's efficacy.

In some embodiments, a composition based on a chewing gum comprises a therapeutically effective amount of an anti-viral active compound, a sugar alcohol, a blend of sugar alcohols, a gum base, or combinations thereof. In certain embodiments, a therapeutically effective amount of anti-viral agents comprises about 0.1% to about 20% by weight, based on the total weight of the composition. Examples of anti-viral active compounds include, but are not limited to, chitosan, zinc, potassium iodide, povidone iodine, chlorhexidine, cetylpyridinium chloride, fucoidan, giloy, quinine, plant enzymes, plant proteins, plant essential oils, ACE-2 inhibitors, anti-viral drug compounds, Cannabis sativa extracts from either hemp or marijuana, molnupiravir, chemokines, cytokines, immune stimulating agents, immune modulating agents, enzymes, ribavirin, protease inhibitors, helicase inhibitors, polymerase inhibitors, helicase inhibitors, neuraminidase inhibitors, nucleoside reverse transcriptase inhibitors, non-nucleoside reverse transcriptase inhibitors, purine nucleosides, chemokine receptor antagonists, interleukins, Althaea officinalis, Commiphora molmol, Glycyrrhiza glabra, Hedera helix, Sambucus nigra, Allium sativum, Andrographis paniculata, Echinacea angustifolia, Echinacea purpurea, Eucalyptus globulus essential oil, Justicia pectoralis, Magnolia officinalis, Mikania glomerata, Pelargonium sidoides, Pimpinella anisum, Salix sp, Zingiber officinale or combinations thereof.

Examples of ACE-2 inhibitors comprise: benazepril (LOTENSIN®, LOTENSIN HCT®), captopril (CAPOTEN®), enalapril (VASOTEC®), fosinopril (MONOPRIL®), lisinopril (PRINIVIL®, ZESTRIL®), moexipril (UNIVASC®), perindopril (ACEON®), quinapril (ACCUPRIL®), ramipril (ALTACE®), and trandolapril (MAVIK®).

In certain embodiments, the non-nucleoside reverse transcriptase inhibitor (NNRTI) analogs, variants or combinations thereof, comprises: etravirine, efavirenz, nevirapine, rilpivirine, delavirdine, or nevirapine. In certain embodiments, a nucleoside reverse transcriptase inhibitor (NRTI), comprises: lamivudine, zidovudine, emtricitabine, abacavir, zalcitabine, dideoxycytidine, azidothymidine, tenofovir disoproxil fumarate, didanosine (ddI EC), dideoxyinosine, stavudine, abacavir sulfate or combinations thereof.

Cannabinoids in the chewing gum composition according to embodiments may be synthetic or procured from natural source. Natural sources of cannabinoids may be from cannabis plants, hemp plants, or other organisms capable of producing cannabinoids. Organisms capable of producing cannabinoids may be genetically modified. Where cannabinoids are from natural sources, a combination of cannabinoids may be present at different concentration. The sources may be chosen such that a cannabinoid may be present as the major cannabinoid, such as CBD, CBG, or THC.

Synthetic cannabinoids may be synthesized by methods known in the art. Synthetic cannabinoids are purer, such that only one cannabinoid may be present. A combination of cannabinoids may be provided at ratios as desired. This may be done to achieve the desired concentrations for the various synthetic cannabinoids.

In certain embodiments, cannabinoids may be provided in a solid material composed of an edible solid, such as a sugar alcohol, to prevent binding with the gum base. Other solids suitable for embedding cannabinoids are contemplated, such that cannabinoids or derivatives thereof are provided within internal voids of solid materials. Alternatively, cannabinoids or derivatives thereof may be provided in a granule embedded into the gum matrix. Cannabinoids or derivatives thereof provided in these manners may improve cannabinoid release during mastication of the chewing gum according to embodiments.

Other suitable carriers which may be combined with cannabinoids before inclusion into the gum matrix may include certain celluloses such as microcrystalline cellulose derivatives, dextran, agarose, agar, pectin, alginate, xanthan, chitosan, or starch. The combination of cannabinoids and suitable carriers may result in cannabinoids being present within internal voids of these carriers.

Providing cannabinoids by combining with a suitable carrier or by providing cannabinoids in a capsule within the gum matrix may enable controlled release of cannabinoids during chewing of the chewing gum composition.

In certain embodiments, cannabinoids or derivatives thereof may also be provided in microencapsulated or nanoencapsulated form or in freeze dried form. Microencapsulated, nanoencapsulated, or freeze-dried cannabinoids may improve the chewing gum's taste, prevent binding with the gum matrix, control cannabinoid release during mastication, and further improve bioavailability of the cannabinoids.

In the chewing gum composition according to embodiments, cannabinoids may be provided in encapsulated form. Microencapsulation or nanoencapsulation into particles may improve bioavailability profiles of cannabinoids. Encapsulation of cannabinoids may result in particles of size 20-40 nm. Microencapsulation or nanoencapsulation may be by liposomal encapsulation, such that the cannabinoids are present inside particles having lipid walls. Other encapsulation methods may be used.

In certain embodiments, freeze dried cannabinoids may be in solid form obtained from freezing cannabis oil containing cannabinoids and subliming other components, leaving a solid having a high cannabinoid concentration. Solid cannabinoids may be effectively incorporated into a chewing composition by combining with other suitable solid carriers and embedding the resulting solid as a granule within the chewing gum composition.

In other embodiments the composition comprises: about 10% to about 80% by weight based on the total weight of the composition, of a sugar, sugar blend, sugar alcohol, or a blend of sugar alcohols, and, about 5% to about 80% by weight based on the total weight of the composition, of a gum base. In certain embodiments, the composition further comprises: flavoring, tableting lubricants and powder flow agents, intensive sweeteners, sugar substitutes or combinations thereof. In certain embodiments, the composition comprises about 1% to about 20% by weight of flavoring, about 0.1% to about 10% by weight of tableting lubricants and powder flow agents, about 0.01% to about 2% by weight of intensive sweeteners and/or sugar substitutes. In embodiments, the sugar or sugar blend comprise dextrose, sucrose, fructose, glucose or combinations thereof. In other embodiments, the sugar alcohol or sugar alcohol blend comprise: sorbitol, isomalt, xylitol, maltitol, mannitol, erythritol, allulose, lactose or combinations thereof. In other embodiments, a sugar substitute comprises stevia, sucralose, monk fruit, honey or agave nectar. In certain embodiments, the chewing gum composition comprises a flavoring agent, e.g., fruity flavors, menthol flavor, eucalyptus, mint flavor, peppermint flavor, spearmint flavor, and the like. Flavorings can be in the form of flavored extracts, volatile oils, chocolate flavorings, peanut butter flavoring, cookie crumbs, crisp rice, vanilla or any commercially available flavoring. Examples of useful flavoring include, but are not limited to, pure anise extract, imitation banana extract, imitation cherry extract, chocolate extract, pure lemon extract, pure orange extract, pure peppermint extract, imitation pineapple extract, imitation rum extract, imitation strawberry extract, or pure vanilla extract; or volatile oils, such as balm oil, bay oil, bergamot oil, cedarwood oil, walnut oil, cherry oil, cinnamon oil, clove oil, or peppermint oil; peanut butter, chocolate flavoring, vanilla cookie crumb, butterscotch or toffee.

An elastomeric base is normally present in the chewing gum composition in an amount of about 25 to about 85% by weight, based on the total weight of the chewing gum composition.

Other suitable carriers which may be combined with the anti-viral agents before inclusion into the gum matrix may include certain celluloses such as microcrystalline cellulose derivatives, dextran, agarose, agar, pectin, alginate, xanthan, chitosan, tri-calcium phosphate, inulin, calcium carbonate or starch.

Lozenges. In another embodiment, the composition is a lozenge which releases the active agent(s) over a period of time, e.g., from about 3 minutes or more, once it is in the subject's mouth. The lozenges are formulated to administer a dose of about 1 to 500 mg of the active agent per application directly to the oral mucosa inside the mouth.

Accordingly, in certain embodiments, a composition for a lozenge comprises a sugar, a sugar blend, sugar alcohol, a blend of sugar alcohols, sweeteners, a bulk filler, and/or derivatives thereof, flavorings, tableting lubricants, powder flow agents or combinations thereof. In certain embodiments, the composition comprises, based on the total weight of the composition: about 10% to about 80% by weight of a sugar, a sugar blend, sugar alcohol, blend of sugar alcohols, sweetener, about 5% to about 50% by weight of bulk filler, about 0.1% to about 10% by weight of a flavor powder, about 0.1% to about 10% by weight of tableting lubricants and powder flow agents, or combinations thereof.

In one embodiment, the composition, based on the total weight of the composition, comprises: about 55% to about 70% by weight of a sugar, a sugar blend, sugar alcohol, blend of sugar alcohols, sweetener, about 5% to about 40% by weight of bulk filler, about 0.1% to about 5% by weight of a flavor powder, about 0.1% to about 5% by weight of tableting lubricants and powder flow agents, or combinations thereof. In certain embodiments, the sugar or sugar blend comprise dextrose, sucrose, fructose, glucose or combinations thereof. In other embodiments, the sugar alcohol or sugar alcohol blend comprise: sorbitol, isomalt, xylitol, maltitol, mannitol, erythritol or combinations thereof and the sweetener comprises stevia, sucralose, monk fruit, honey or agave nectar.

In one embodiment, the tablet flow agent comprises magnesium stearate.

In other embodiments, a bulk filler comprises microcrystalline cellulose (MCC), bamboo fibers, tri-calcium phosphate, inulin, calcium carbonate or combinations thereof. In order to manufacture a slower versus fast-dissolving lozenge or tablet, the proportion of the bulk fillers are increased or decreased relative to the other constituents to alter the dissolution rate of the lozenge, i.e., fast-dissolving, slow dissolving etc. The bulk fillers absorb moisture quickly which creates the dissolution. Suitable fillers include celluloses and cellulose derivatives including microcrystalline cellulose, hydroxypropylcellulose and sodium carboxymethylcellulose, lactose, starches including potato starch and corn starch, carbohydrates including a cellulose derivative, e.g., hemicellulose. The cellulose derivative may be of natural origin, e.g., dextran, agarose, agar, pectin, alginate, xanthan, chitosan, starch. The cellulose derivative may also be of synthetic or semi-synthetic origin. In certain embodiments, a bulk filler comprises microcrystalline cellulose (MCC), bamboo fibers, or combinations thereof. The bulk fillers are present in the composition from about 5% to about 50% by weight of bulk filler, based on total weight of the composition. Specific examples of a suitable microcrystalline cellulose is microcrystalline cellulose comprising: AVICEL™ grades PH-100, PH-102, PH-103, PH-105, PH-112, PH-113, PH-200, PH-300, PH-302, VIVACEL™ grades 101, 102, 12, 20 and EMOCEL™ grades 50M and 90M, and the like, and mixtures thereof.

Flavors, coloring agents, spices, and the like can be incorporated into the product. Flavorings can be in the form of flavored extracts, volatile oils, chocolate flavorings, peanut butter flavoring, cookie crumbs, crisp rice, vanilla or any commercially available flavoring. Examples of useful flavoring include, but are not limited to, pure anise extract, imitation banana extract, imitation cherry extract, chocolate extract, pure lemon extract, pure orange extract, pure peppermint extract, imitation pineapple extract, imitation rum extract, imitation strawberry extract, or pure vanilla extract; or volatile oils, such as balm oil, bay oil, bergamot oil, cedarwood oil, walnut oil, cherry oil, cinnamon oil, clove oil, or peppermint oil; peanut butter, chocolate flavoring, vanilla cookie crumb, butterscotch or toffee.

Other formulations. The active agents may further be formulated with acceptable excipients and/or carriers for oral consumption. The carrier may be a liquid, gel, gelcap, capsule, powder, solid tablet (coated or non-coated), tea, or the like. Suitable excipient and/or carriers include maltodextrin, calcium carbonate, dicalcium phosphate, tricalcium phosphate, microcrystalline cellulose, dextrose, rice flour, magnesium stearate, stearic acid, croscarmellose sodium, sodium starch glycolate, crospovidone, sucrose, vegetable gums, lactose, methylcellulose, povidone, carboxymethylcellulose, corn starch, and the like (including mixtures thereof). Preferred carriers further include calcium carbonate, magnesium stearate, maltodextrin, and mixtures thereof. The various ingredients and the excipient and/or carrier are mixed and formed into the desired form using conventional techniques. The tablet or capsule of the present invention may be coated with an enteric coating that dissolves at a pH of about 6.0 to 7.0. A suitable enteric coating that dissolves in the small intestine but not in the stomach is cellulose acetate phthalate. Further details on techniques for formulation for and administration may be found in the latest edition of Remington's Pharmaceutical Sciences (Mack Publishing Co., Easton, Pa.). Such formulations may preferably comprise from about 1 mg to 500 mg of the concentrate. Where the formulation is an oral delivery vehicle such as a capsule or tablet, the oral delivery vehicle may comprise from about 1 to 250 mg of the concentrate, 10 to 200 mg of the concentrate to 10 to 100 mg of the concentrate. A daily dosage may comprise 1, 2, 3, 4 or 5 of the oral delivery vehicles.

In other embodiments, the active agents are provided as a powder or liquid suitable for adding by the consumer to a food or beverage. For example, in some embodiments, the concentrate can be administered to an individual in the form of a powder, for instance to be used by mixing into a beverage, or by stirring into a semi-solid food such as a pudding, topping, sauce, puree, cooked cereal, or salad dressing, for instance, or by otherwise adding to a food.

In other embodiments, the compositions comprising one or more additional bioactive agents, phytonutrients, or nutraceutical agents to provide a dietary supplement. For example, the dietary supplement of the present invention may also contain optional ingredients including, for example, herbs, vitamins, minerals, enhancers, colorants, sweeteners, flavorants, inert ingredients, and the like. For example, the dietary supplement of the present invention may contain one or more of the following: ascorbates (ascorbic acid, mineral ascorbate salts, rose hips, acerola, and the like), dehydroepiandosterone (DHEA), Fo-Ti or Ho Shu Wu (herb common to traditional Asian treatments), Cat's Claw (ancient herbal ingredient), green tea (polyphenols), inositol, kelp, dulse, bioflavinoids, maltodextrin, nettles, niacin, niacinamide, rosemary, selenium, silica (silicon dioxide, silica gel, horsetail, shavegrass, and the like), spirulina, zinc, and the like. Such optional ingredients may be either naturally occurring or concentrated forms. Nutraceutical agents are natural, bioactive chemical compounds that have health promoting, disease preventing or medicinal properties. Examples of nutraceutical agents that may be combined with the concentrates of the present invention include, but are not limited to, resveratrol, fucoidan, Allium cepa, Allium sativum, Aloe vera, Angelica Species, Naturally Occurring Antioxidants, Aspergillus oryzae, barley grass, Bromelain, Carnitine, carotenoids and flavonoids, Catechin, Centella asiatica (Gotu kola), Coenzyme Q10, Chinese Prepared Medicines, Coleus forskohlii, Commiphora mukul, Conjugated Linoleic Acids (CLAs), Crataegus oxyacantha (Hawthorne), Curcuma longa (Turmeric), Echinacea Species (Purple Coneflower), Eleutherococcus senticosus (Siberian Ginseng), Ephedra Species, Dietary Fish Oil, Genistein, Ginkgo biloba, Glycyrrhiza (Licorice), Hypericum perforatum (St. John's Wort), Hydrastis (Goldenseal) and other Berberine-containing plants, Lactobacillus, Lobelia (Indian Tobacco), Melaleuca alternifolia, Menaquinone, Mentha piperita, n-glycolylneuraminic acid (NGNA), Panax ginseng, Pancreatic Enzymes, Piper mythisticum, Procyanidolic Oligomers, Pygeum africanum, Quercetin, Sarsaparilla species, Serenoa repens (Saw palmetto, Sabal serrulata), Silybum marianum (Milk Thistle), Rosemary/Lemon balm, Selenite, Tabebuia avellanedae (LaPacho), Taraxacum officinale, Tanacetum parthenium (Feverfew), Taxol, Uva ursi (Bearberry), Vaccinium myrtillus (Blueberry), Valerian officinalis, Viscum album (Mistletoe), Vitamin A, Beta-Carotene and other carotenoids, and Zingiber officinale (Ginger).

In some embodiments, the dietary supplements further comprise vitamins and minerals including, but not limited to, calcium phosphate or acetate, tribasic; potassium phosphate, dibasic; magnesium sulfate or oxide; salt (sodium chloride); potassium chloride or acetate; ascorbic acid; ferric orthophosphate; niacinamide; zinc sulfate or oxide; calcium pantothenate; copper gluconate; riboflavin; beta-carotene; pyridoxine hydrochloride; thiamin mononitrate; folic acid; biotin; chromium chloride or picolonate; potassium iodide; sodium selenate; sodium molybdate; phylloquinone; vitamin D3; cyanocobalamin; sodium selenite; copper sulfate; vitamin A; vitamin C; inositol; potassium iodide. Suitable dosages for vitamins and minerals may be obtained, for example, by consulting the U.S. RDA guidelines.

Manufacture

The various compositions embodied herein can be manufactured using known methods.

The manufacturing of the various compositions utilizes methods of tablet compression incorporating ingredients in powder forms. The method for manufacturing the chewing gum combines the powdered ingredients with gum bases into a mixture that is then milled into a powder with a certain particle size. The powdered gum composition is compressed into a tablet using tablet presses. The method for manufacturing lozenges combines the powdered ingredients into a mixture that is then compressed into a tablet using tablet presses. The chewing gum may be compressed into either a single layer, a bi-layer or a tri-layer tablet.

Effective Doses

Effective doses of the compositions of the present invention, for the treatment of the above-described diseases, vary depending upon many different factors, including means of administration, physiological state of the patient, whether the patient is human or an animal, other medications administered, and whether treatment is prophylactic or therapeutic. Usually, the patient is a human.

The compositions can be administered on multiple occasions, wherein intervals between single dosages can be as-needed, hourly, daily, weekly, monthly, or yearly. Dosage and frequency may vary depending on the half-life of the compounds of the invention. In therapeutic applications, a relatively high dosage at relatively short intervals is sometimes required until progression of the disease is reduced or terminated, and sometimes until the patient shows partial or complete amelioration of symptoms of the disease. Thereafter, the patient can be administered a prophylactic regime.

For any active agent used in the methods of the invention, the therapeutically effective amount or dose can be estimated initially from activity assays in cell cultures and/or animals.

The pharmaceutical compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are prepared according to conventional mixing, granulating, or coating methods, and typically contain about 0.1% to 75%, preferably about 1% to 50%, of the active ingredient.

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above-described embodiments.

All publications and patent documents cited in this application are incorporated by reference for all purposes to the same extent as if each individual publication or patent document were so individually denoted. By their citation of various references in this document, applicants do not admit any particular reference is “prior art” to their invention.

### REFERENCES

1. Mythily Srinivasan, Susan L Zunt, Lawrence I Goldblatt; Oral epithelial expression of angiotensin converting enzyme-2: Implications for COVID-19 diagnosis and prognosis, bioRxiv 2020.06.22.165035
2. Krishna Sriam, Paul Insel, Rohit Loomba; What is the ACE2 receptor, how is it connected to coronavirus and why might it be key to treating COVID-19? The experts explain, TheConversation.com, 2021.

What is claimed is:

1. A composition comprising: an active ingredient, a sugar alcohol, a blend of sugar alcohols, a sweetener, flavorings, a gum base, or combinations thereof.

2. The composition of claim 1, wherein the active ingredient is an anti-viral agent or an angiotensin-converting enzyme (ACE) inhibitor.

3. The composition of claim 1, wherein the composition further comprises a cannabinoid or derivatives thereof.

4. The composition of claim 1, wherein the anti-viral agent comprises: chitosan, zinc, potassium iodide, chlorhexidine, cetylpyridinium chloride, fucoidan, giloy, quinine, povidone-iodine, hydrogen peroxide, carbamide peroxide, plant proteins, cyclodextrin, Citrox, essential oils, angiotensin-converting enzyme (ACE) inhibitors, or in combinations thereof.

5. The composition of claim 2, wherein the ACE inhibitors comprise: benazepril, captopril, enalapril, fosinopril, lisinopril, moexipril, perindopril, quinapril, ramipril, trandolapril or combinations thereof.

6. The composition of claim 2, wherein the anti-viral agent comprises from about 1.0% to about 10.0% by weight, based on the total weight of the composition.

7. The composition of claim 3, wherein the cannabinoid or derivatives thereof comprise from about 1.0% to about 10.0% by weight, based on the total weight of the composition.

8. The composition of claim 1, wherein the composition, based on the total weight of the composition, comprises:
about 40% to about 80% by weight of a sugar alcohol or a blend of sugar alcohols, or a sweetener or a combination thereof;
about 20.0% to about 30.0% by weight of a gum base;
about 1% to about 10% by weight of an anti-viral agent or combination of anti-viral agents,
about 2% to about 15% by weight of a flavoring in liquid or powder form;
about 1% to about 5% by weight of tableting lubricants and powder flow agents;
about 0.2% to about 0.6% by weight of intensive sweeteners; or
combinations thereof.

9. A composition comprising: sugar alcohol or a blend of sugar alcohols, a flavoring(s), an active agent(s), tableting lubricants, powder flow agents, intensive sweeteners or combinations thereof.

10. The composition of claim 9, wherein the blend of sugar alcohols comprise one or more of: sorbitol, isomalt, xylitol, maltitol, mannitol or erythritol.

11. The composition of claim 9, wherein the active agent(s) comprise at least one of: an anti-viral agent, derivatives thereof or combinations thereof.

12. The composition of claim 9, wherein the active agents based on the total weight of the composition, comprise about 1.0% to about 10.0% by weight.

13. The composition of claim 9, wherein the flavoring is in a liquid and/or powder form.

14. The composition of claim 9, wherein the flavoring, based on the total weight of the composition, comprise about 2.0% to about 12.0% by weight.

15. The composition of claim 9, wherein the sugar alcohol or a blend of sugar alcohols based on the total weight of the composition, comprise about 70.0% to about 90.0% by weight.

16. The composition of claim 9, wherein the tableting lubricants and powder flow agents, based on the total weight of the composition, comprise about 1.5% to about 5.0% by weight.

17. The composition of claim 9, wherein the active ingredient is an anti-viral agent or an angiotensin-converting enzyme (ACE) inhibitor.

18. The composition of claim 9, wherein the anti-viral agent comprises from about 1.0% to about 10.0% by weight, based on the total weight of the composition.

19. The composition of claim 9, wherein the composition further comprises a cannabinoid or derivatives thereof.

20. The composition of claim 19, wherein the cannabinoid or derivatives thereof comprise from about 1.0% to about 10.0% by weight, based on the total weight of the composition.

21. A composition comprising: a gum base, a sugar alcohol, a blend of sugar alcohols, sweeteners, a bulk filler, an anti-viral agent or combination of anti-viral agents, flavorings, tableting lubricants, powder flow agents or combinations thereof.

22. The composition of claim 21, wherein the composition, based on the total weight of the composition, comprises:
about 10% to about 80% by weight of a sugar alcohol, blend of sugar alcohols, sweetener,
about 5% to about 50% by weight of bulk filler,
about 1% to about 10% by weight of an anti-viral agent or combination of anti-viral agents,
about 0.1% to about 10% by weight of a flavor powder,
about 0.1% to about 10% by weight of tableting lubricants and powder flow agents,
or combinations thereof.

23. The composition of claim 21, wherein the composition, based on the total weight of the composition, comprises:
about 55% to about 70% by weight of a sugar, a sugar blend, sugar alcohol, blend of sugar alcohols, sweetener,
about 5% to about 40% by weight of bulk filler,
about 1% to about 10% by weight of an anti-viral agent or combination of anti-viral agents,
about 0.1% to about 5% by weight of a flavor powder,
about 0.1% to about 5% by weight of tableting lubricants and powder flow agents,
or combinations thereof.

24. The composition of claim 21, wherein the sugar or sugar blend comprise dextrose, sucrose, fructose, glucose or combinations thereof.

25. The composition of claim 23, wherein the sugar alcohol or sugar alcohol blend comprise: sorbitol, isomalt, xylitol, maltitol, mannitol, erythritol or combinations thereof.

26. The composition of claim 23, wherein the sweetener comprises stevia, sucralose, monk fruit, honey or agave nectar.

27. The composition of claim 21, wherein the tablet flow agent comprises magnesium stearate.

28. The composition of claim 21, wherein a bulk filler comprises microcrystalline cellulose (MCC), bamboo fibers, or combinations thereof.

29. A composition consisting of: a sugar, a sugar blend, sugar alcohol, a blend of sugar alcohols, sweeteners, a bulk filler, an anti-viral agent or combination of anti-viral agents, flavorings, tableting lubricants, powder flow agents and combinations thereof.

30. The composition of claim 29, wherein the composition, based on the total weight of the composition, consists of:
about 55% to about 70% by weight of a sugar, a sugar blend, sugar alcohol, blend of sugar alcohols, sweetener,
about 5% to about 40% by weight of bulk filler,
about 1% to about 10% by weight of an anti-viral agent or combination of anti-viral agents,
about 0.1% to about 5% by weight of a flavor powder,
about 0.1% to about 5% by weight of tableting lubricants and/or powder flow agents,
and combinations thereof.

31. The composition of claim 29, wherein the composition further comprises a cannabinoid or derivatives thereof.

32. The composition of claim 31, wherein the cannabinoid or derivatives thereof comprise from about 1.0% to about 10.0% by weight, based on the total weight of the composition.

33. A method of preventing or treating diseases or disorders associated with viruses in a subject, comprising: administering to the subject a composition comprising: an anti-viral agent or combination of anti-viral agents, sugar alcohol, a blend of sugar alcohols, a gum base, or combinations thereof.

34. The method of claim 33, wherein the composition, based on the total weight of the composition, comprises:
about 10% to about 80% by weight of a sugar, sugar blend, sugar alcohol, or a blend of sugar alcohols,
about 5% to about 80% of a gum base.

35. The method of claim 33, further comprising: flavoring, tableting lubricants and powder flow agents, intensive sweeteners, sugar substitutes or combinations thereof.

36. The method of claim 33, wherein the composition, based on the total weight of the composition, further comprises:
about 1% to about 20% by weight of flavoring,
about 0.1% to about 10% by weight of tableting lubricants and powder flow agents,
about 0.01% to about 2% by weight of intensive sweeteners.

37. The method of claim 33, wherein the sugar or sugar blend comprise dextrose, sucrose, fructose, glucose or combinations thereof.

38. A method of preventing or treating diseases or disorders associated with viruses in a subject, comprising: administering to a subject in need thereof, a gum-based composition for chewing, the composition comprising, based on the total weight of the composition:
about 10% to about 80% by weight of a sugar, sugar blend, sugar alcohol, or a blend of sugar alcohols,
about 5% to about 80% of a gum base,
about 1% to about 10% by weight of an anti-viral agent or combination of anti-viral agents,
about 1% to about 20% by weight of flavoring,
about 0.1% to about 10% by weight of tableting lubricants and powder flow agents,
about 0.01% to about 2% by weight of intensive sweeteners,
or combinations thereof.

39. The method of claim 38, wherein the composition is in a gum or tablet form.

40. A composition comprising: an anti-viral agent or combination of anti-viral agents, a sugar, sugar blend, sugar alcohol, a blend of sugar alcohols, a gum base, or combinations thereof.

41. The composition of claim 40, wherein the composition, based on the total weight of the composition, comprises:
about 1% to about 10% by weight of an anti-viral agent or combination of anti-viral agents,
about 10% to about 80% by weight of a sugar, sugar blend, sugar alcohol, or a blend of sugar alcohols,
about 5% to about 80% of a gum base.

42. The composition of claim 40, further comprising: flavoring, tableting lubricants and powder flow agents, intensive sweeteners, sugar substitutes or combinations thereof.

43. The composition of claim 40, wherein the composition, based on the total weight of the composition, further comprises:
about 1% to about 20% by weight of flavoring,
about 0.1% to about 10% by weight of tableting lubricants and powder flow agents,
about 0.01% to about 2% by weight of intensive sweeteners.

44. The composition of claim 41, wherein the sugar alcohol or sugar alcohol blend comprise: sorbitol, isomalt, xylitol, maltitol, mannitol, erythritol or combinations thereof.

45. The composition of claim 42, wherein a sugar substitute comprises stevia, sucralose, monk fruit, honey or agave nectar.

46. A composition consisting of: an anti-viral agent or combination of anti-viral agents, a sugar alcohol or a blend of sugar alcohols, tableting lubricants, powder flow agents and intensive sweeteners.

47. The composition of claim 46, wherein the composition, based on the total weight of the composition, consists of:
about 70.0% to about 90.0% by weight of a sugar, sugar blend, sugar alcohol, or a blend of sugar alcohols,
about 1% to about 10% by weight of an anti-viral agent or combination of anti-viral agents,
about 2% to about 12% by weight of flavoring,
about 1% to about 5% by weight of tableting lubricants and powder flow agents,
about 0.1% to about 2% by weight of intensive sweeteners.

48. The composition of claim 47, wherein the composition further comprises a cannabinoid or derivatives thereof.

49. The composition of claim 48, wherein the cannabinoid or derivatives thereof comprise from about 1.0% to about 10.0% by weight, based on the total weight of the composition.

50. A composition consisting of: an anti-viral agent or combination of anti-viral agents, a sugar alcohol or a blend of sugar alcohols, or derivatives thereof, gum base, tableting lubricants, powder flow agents and intensive sweeteners.

51. The composition of claim 50, wherein the composition, based on the total weight of the composition, consists of:
about 42.0% to about 80.0% by weight of a sugar, sugar blend, sugar alcohol, or a blend of sugar alcohols,
about 20% to about 30% of a gum base,
about 1% to about 10% by weight of an anti-viral agent or combination of anti-viral agents,
about 2% to about 12% by weight of flavoring,
about 1% to about 5% by weight of tableting lubricants and powder flow agents,
about 0.2% to about 0.6% by weight of intensive sweeteners.

52. The composition of claim 50, wherein the composition further comprises a cannabinoid or derivatives thereof.

53. The composition of claim 52, wherein the cannabinoid or derivatives thereof comprise from about 1.0% to about 10.0% by weight, based on the total weight of the composition.
